(19) United States
(12) Patent Application Publication
(10) Pub. No.: US 2004/0242542 A1
(43) Pub. Date: Dec. 2, 2004

(54) Compositions and methods for inducing CD81 dependent antiproliferation and for treating hepatitis C and other disorders

(75) Inventors: Kenneth J. Shea, Irvine, CA (US); Carl E. Wagner, Irvine, CA (US)

Correspondence Address: Robert D. Buyan, Stout, Uxa, Buyan & Mullins, LLP, Suite 300, 4 Venture, Irvine, CA, 92618, US

(73) Assignee: The Regents of the University of California, Oakland, CA

(21) Appl. No.: 10/810,497

(22) Filed: Mar. 25, 2004

Related U.S. Application Data
(60) Provisional application No. 60/457,854, filed on Mar. 25, 2003.

(51) Int. Cl. A61K 31/69; A61K 31/66; A61K 31/505; A61K 31/445

(52) U.S. Cl. 514/64; 514/319; 514/408; 514/553; 514/114

(57) ABSTRACT

Small molecule analogues of 1-aminoadamantane and methods for using such compositions to induce CD81-dependent antiproliferative effects and/or for the treatment of disorders of human or veterinary patients and/or for the manufacture of therapeutic preparations used to treat such disorders.

### RELATED APPLICATION

This patent application claims priority to U.S. Provisional patent Application No.60/457,854 entitled “Compositions and Methods for Inducing CD81 Dependent Antiproliferation and for Treating Hepatitis C and Other Disorders” filed on Mar. 25, 2003, the entirety of which is expressly incorporated herein by reference.

### FIELD OF THE INVENTION

The present invention relates generally to the fields of organic chemistry and pharmacology, and more particularly to the composition of various small molecule analogues of 1-aminoadamantane and methods for using such compositions to induce CD81-dependent antiproliferative effects and/or for the treatment of disorders of human or veterinary patients and/or for the manufacture of therapeutic preparations used to treat such disorders.

### BACKGROUND OF THE INVENTION

The hepatitis C virus (HCV) affects a substantial part of the world's population, from 1-2%, and complications arising from the progression of the chronic infection of the liver include cirrhosis and liver cancer. Currently, treatment of HCV with _-interferons and ribavirin affects a continued remittance of viral RNA in less than 50% of treated patients. Recent studies for the treatment of hepatitis C with amantadine alone and in combination with alpha-interferon and ribavirin have shown promise in the reduction of alanine aminotransferase (ALT), the enzyme released by the lysis of damaged liver cells, and HCV RNA levels. Other methods of treatment currently under investigation involve viral protease inhibitors, but those developed thus far include only a small variety of peptide substrates and analogues. In view of the difficulty of administering small peptides as therapeutic drugs, and the promise shown by drugs like amantadine, there exists a need for the synthesis and development of new small molecule analogues of 1 -aminoadamantane which bind to and/or activate CD81 and/or which exhibit therapeutic activity against hepatitis C or other disorders that involve CD81 antiproliferation.

### SUMMARY OF THE INVENTION

The present invention provides novel amine complexes with 1-boraadamatane, a number of which exhibit antiproliferative effects on CD81-enriched cell lines to provide evidence for binding and activation of CD81. CD81 is a member of the tetraspanin family of membrane proteins found in all cell lineages in the liver. CD81 signals for antiproliferation when bound by antibodies. It is known that the HCV-E2 envelope glycoprotein binds to the CD81 protein. While it is unclear whether virus entry into host cells is directly linked to virus attachment via CD81 for HCV, this step in the viral lifecycle has recently proven to be an effective point of attack for other viruses including HIV and rhinoviruses.

Further in accordance with the present invention, there are provided methods for synthesis of amantadine analogues by appending primary amines to 1-boraadamantane to evaluate such compounds for CD81 dependent antiproliferation of CD81-enriched cell lines (astrocyte) vs. CD81-deficient cell lines (C6 glioma). Specific details of the synthetic methods of the present invention as well as related data is now published in Wagner, Carl E. et al., Synthesis of-1-Boraadamantaneamine Derivatives with Selective Astrocyte vs C6 Glioma Antiproliferative Activity. A Novel Class of Anti-Hepatitis C Agents with Potential to Bind CD81, J.Med.Chem., Vol.46,2823-2833 (2003) and Wagner, Carl E., Syntheis and First Molecular Structure of a Bis-2-spiro-1boraadamantane Dirivative, Organic Letters, Vol. 6, No. 3, 313-316 (2004), copies of which are filed herewith and the entireties of which are expressly incorporated herein by reference.

### DETAILED DESCRIPTION

The following detailed description and the examples provided therein are not intended to exhaustively describe all possible embodiements or examples of the invention and, this, shall not limit the scope of the invention in any way.

The amantadine analogues of the present invention that bind to and activate CD81 may have the potential to prevent or treat HCV infections. Each compound's potential for preventive and therapeutic activity stems from the compound's potential to block viral attachment, virus-cell fusion or virus entry into host cells, or similarly counter potential mechanisms of HCV immune evasion. Out of a library of over 500 compounds including randomly selected small molecules and rationally designed small molecules, only the 1-boraadamantane amine compounds display a significant antiproliferative effect on the CD81-enriched astrocytes relative to the CD81-deficient cell lines. In fact, the 1-boraadamantane 1-aminoadamantane complex (1) shows a dose-dependent, astrocyte selective antiproliferative activity with an EC₅₀=7.6_M consistent with the binding and activation of CD81. Additionally, structurally similar adamantane analogues to the active primary amine 1-boraadamantane complexes identified in this assay were synthesized for testing. Since the standard anti-HCV drug, 1-aminoadamantane hydrochloride (amantadine) only slightly reduces astrocyte proliferation at high concentrations (>100 _M), the 1-boraadamantane amine derivatives present a promising lead in the development of small molecules with potential to bind to CD81.

Due to the versatility with which functionalized primary amines can be appended to the 1-boraadamantane core, as well as the stability of the resulting complexes, 1-boraadamantane represents a promising substrate for modification and pharmacology studies en route to 1-aminoadamantane analogues. In fact, several 1-boraadamantane complexes with amines and other nitrogen containing compounds display a pronounced anti-viral effect against the influenza A and B viruses. Based on the evidence for the potent anti-viral pharmacology of 1-boraadamantane amine complexes and the structural similarities to 1-aminoadamantane, a number of 1-boraadamantane amine complexes were synthesized to be evaluated for a CD81 dependent antiproliferative activity in astrocytes vs. C6 glioma.

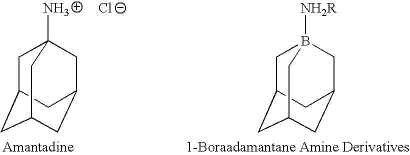

Thus, a number of 1-boraadamantane amine complexes were synthesized. These complexes include complexes 1-5 shown below.

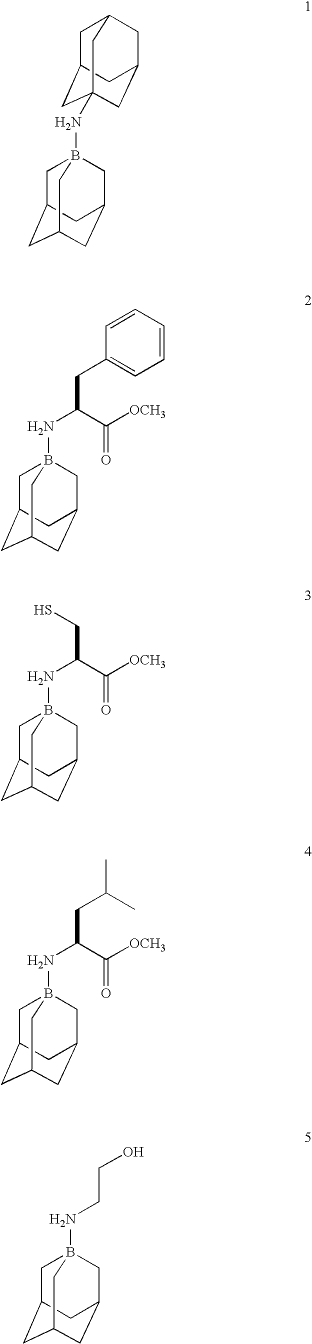

Since the cell based assay identified the complexes of 1-boraadamantane with 1-minoadamantane (1) and L-phenylalanine (2) as the most active compounds for hibiting the proliferation of the astrocyte cell line, several structurally similar amantyl derivatives were synthesized and tested. These structurally similar amantyl derivatives include compounds 6-13 shown below.

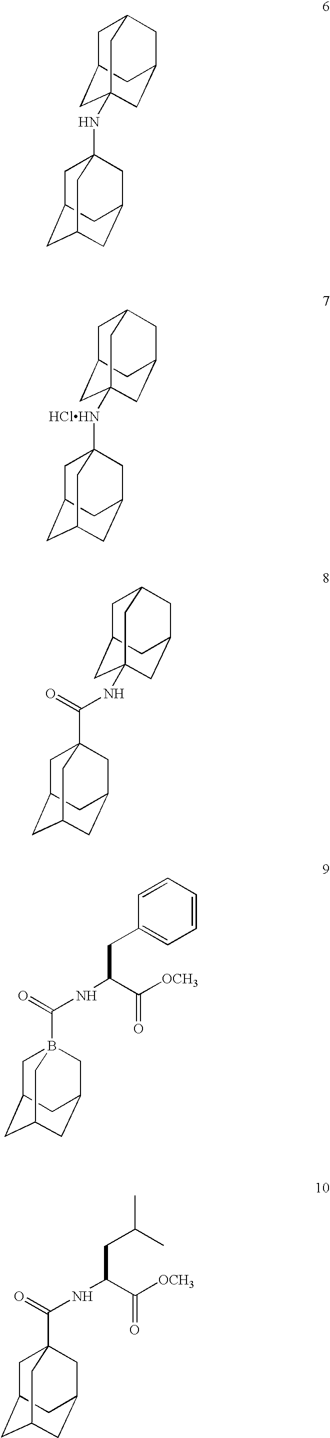

and reduced analog of compound 8.

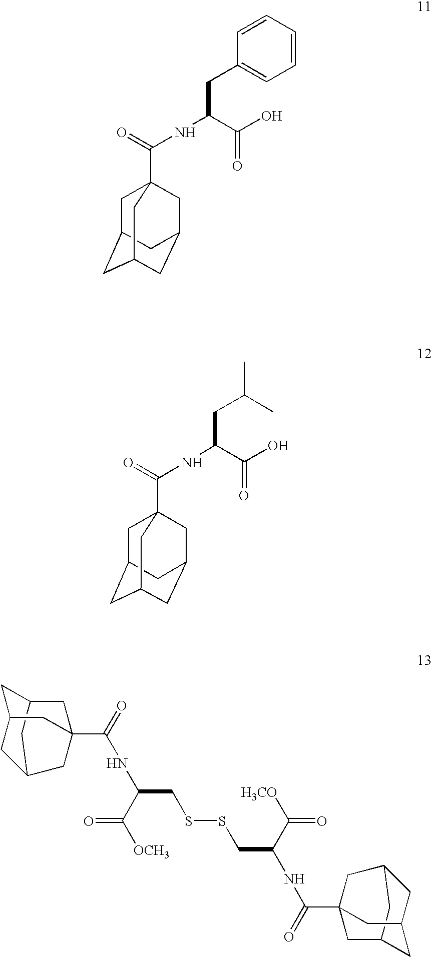

The invention is not limited to only those compounds synthesized. In fact, the theraputic method of the present invention may be carried out by administering to the patient a therapeutically effective amount of any amantadine analogue that has a primary amine appended to 1-boraadamantane, including but not limited to the compounds described above and/or those within General Formula I, as follows:

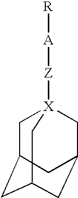

wherein,

X is Boron or Carbon;

A is NH and NHR1, where R1 is H, alkyl or imino-alkyl amino;

Z is a acyclic or cyclic, saturated or unsaturated, chiral or achiral, straight or branched hydrocarbyl group with from 1 to 10 carbon atoms, C═O, SO2, or absent; and

R is an acyclic or cyclic, saturated or unsaturated, chiral or achiral, straight or branched hydrocarbyl group with from 1 to 20 carbon atoms and —CH—R2R3, wherein R2 is selected from aryl, substituted aryl, heteroaryl, substituted heteroaryl, (CH2)n-Q, where n is 1-4 and Q is —SH, —OH, —NH2, —NH—CO—NH2, —NH—C═(NR4) NHR5, —COOH and its alkyl esters, and —CONH2 and R4 and R5 are H, C1-4 alkyl or R4 and R5 may combine to form a cyclic ring, R2 and A may combine to form a cyclic ring; R3 is carboxyl, its alkyl esters, carboxamide or substituted carboxamide, sulfonic acid, sulfonate esters, sulfonamide, substituted sulfonamide, phosphonic and phosphoric acids and their alkyl esters.

### EXAMPLE 1

### Synthesis

1-boraadamantane amine complexes of the present invention may be synthesized by any suitable means. For example, 1-boraadamantane amine complexes may be conveniently derived from 1-boraadamantane·THF (3) in high yield by stoichiometric addition. Thus, 1-boraadamantane·THF (3) was synthesized by the hydroboration of 3-methoxy-7-(methoxymethyl)-3-borabicyclo-[3.3.1]non-6-ene (2). This compound was prepared by the reaction of triallylborane (1) with methyl propargyl ether followed by methanolysis, according to the procedure of Mikhailov and co-workers (Scheme 27). Triallylborane (1) was synthesized according to a method previously reported by Zakharkin and co-workers.

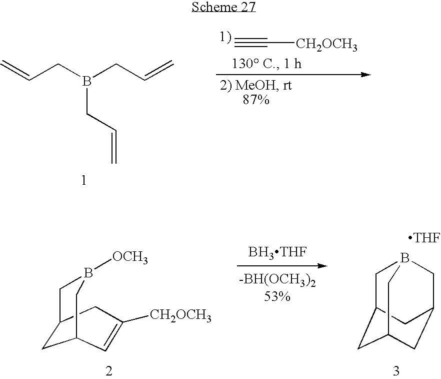

After purification by sublimation, 1-boraadamantane·THF was procured in 46% overall yield from triallylborane.

The first 1-boraadamantane amine complexes to be synthesized and tested included complexes with 1-aminoadamantane (4) and L-phenylalanine methyl ester (5)(Scheme 28).

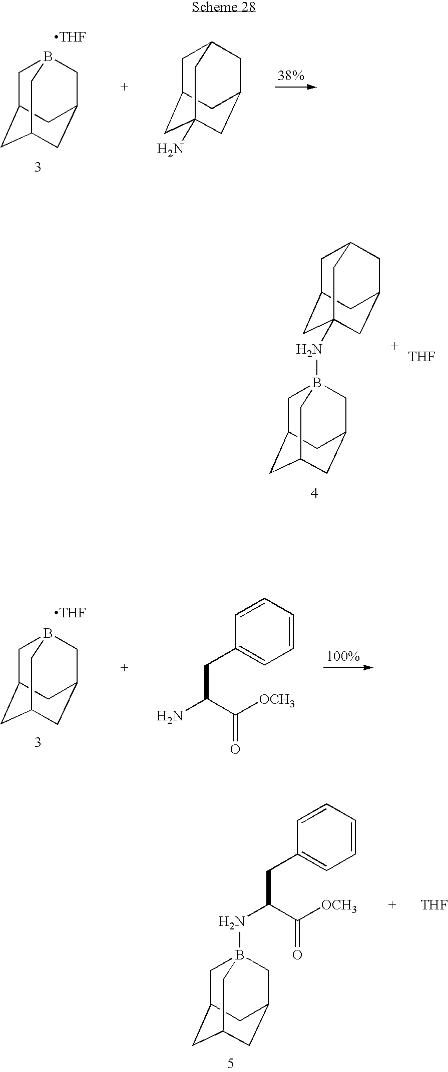

Complex 4 was assembled in 38% yield by addition of a stoichiometric amount of 1-aminoadamantane to 1-boraadamantane·THF and purification by recrystallization in ethanol.

Complex 5 was synthesized in quantitative yield by stoichiometric addition of L-phenylalanine methyl ester to 1-boraadamantane·THF.

Using the same protocol to synthesize amine complex 5 (Scheme 28), complexes of 1-boraadamantane with L-cysteine methyl ester (6) and L-leucine methyl ester (7) were synthesized in quantitative yield. Additionally, a complex of 1-boraadamantane with ethanolamine (8) was also synthesized in quantitative yield for testing.

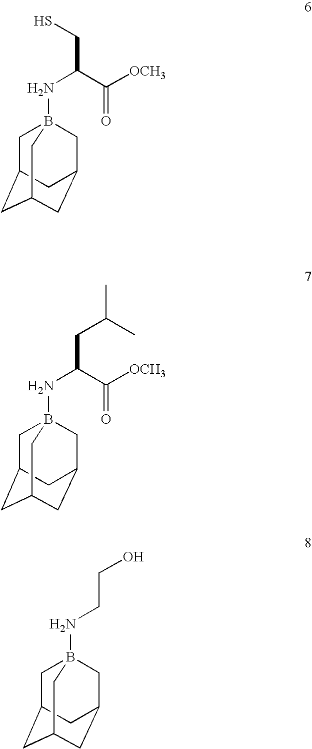

The amine complexes 6, 7 and 8 are air-stable, crystalline complexes. X-ray diffraction was performed on single crystals of complex 5, 6 and 8

The X-ray diffraction study for complex 5 reveals that the amine group of L-phenylalanine methyl ester coordinates to boron in boraadamantane. The crystal structure of 6 establishes that the amine group coordinates to boron rather than the thiol group of L-cysteine methyl ester, and the X-ray crystal structure of 8 reveals that the amine group coordinates to boron rather than the hydroxyl group of ethanolamine. The polarity of the L-cysteine methyl ester as well as the free thiol group improves the solubility of the amino acid methyl ester complex 6 in polar, protic solvents. The compact size of ethanolamine and its free hydroxyl group also improves the solubility of complex 8 in polar, protic solvents. In fact, the solubility of complexes 4-8 in water ranged from 3.8 mg/mL for complex 8 to 0.6 mg/mL for complex 5. With the rare exceptions of triallylboranes, the (C-sp³)—B bonds of trialkylboranes including trimethylborane are stable in water to 100° C. The stability of complex 4 in refluxing ethanol during recrystallization attests to the stability of these complexes in polar, protic solvents.

Several di-adamantyl compounds structurally similar to complex 4 were also prepared. 1-Adamantylcarbonyl chloride was prepared from the reaction of 1-adamantylcarboxylic acid with thionyl chloride, and the acid chloride was subsequently reacted with 1-aminoadamantane to give 1,1-diadamantylamide (9) in 19% yield. Additionally, di-1-adamantylamine (11) and its hydrochloride salt (12) were prepared from 1-bromoadamantane (10) according to the procedure of Mcintyre (Scheme 29).

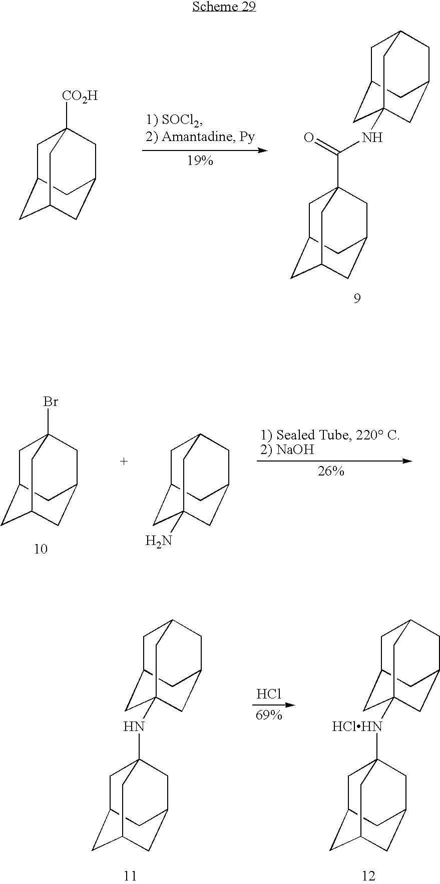

The reaction of 1-bromoadamantane with 1-aminoadamantane in a thick-walled sealed glass tube (220° C., 40 h) furnishes 11 after work up with base in a simple, direct procedure. Di-1-adamantylamine (11) can be protonated with HCI to furnish the hydrochloride salt 12.

Several compounds structurally similar to complexes 5 and 7 were also prepared. 1-Adamantylcarbonyl chloride was reacted with L-phenylalanine methyl ester hydrochloride in methylene chloride and triethylamine (2 equiv) to give amide 13; in 96% yield.

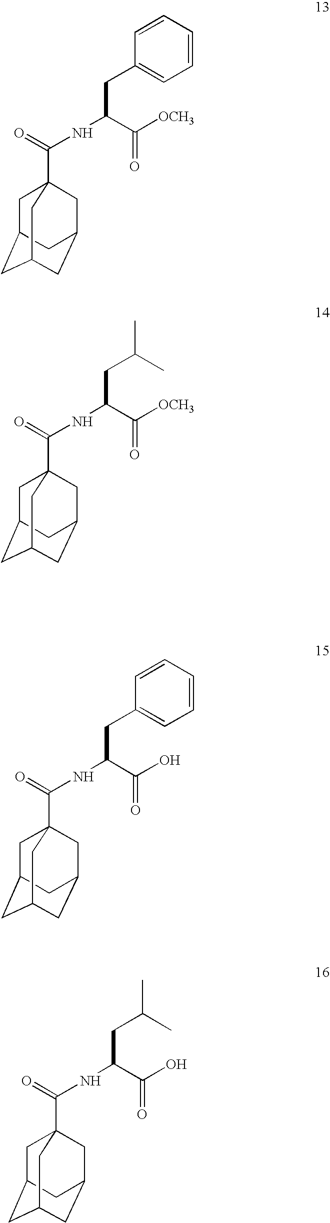

Amide 14 was similarly prepared in 68% yield. Acids 15 and 16 were prepared from the corresponding amides in 63% and 87%, respectively. Due to the versatility with which adamantyl amide analogues can be synthesized by combining the commercially available 1-adamantylcarbonyl chloride with amino-acid methyl and ethyl esters, a series of adamantyl amnide analogues was synthesized. The resulting methyl and ethyl esters in all but one case were readily converted to the corresponding acids by saponification with lithium hydroxide as shown in (Scheme30).

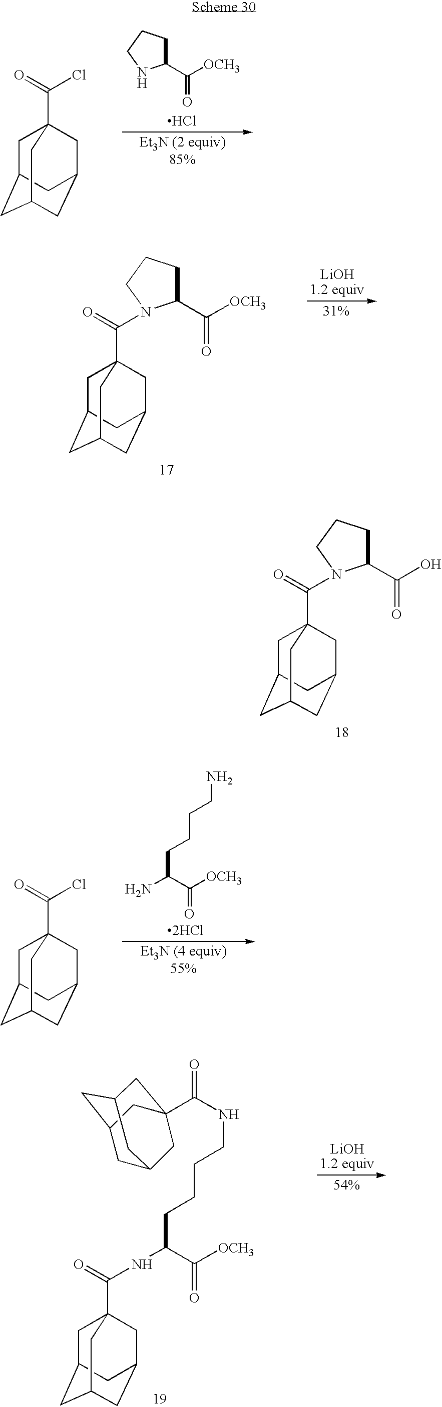

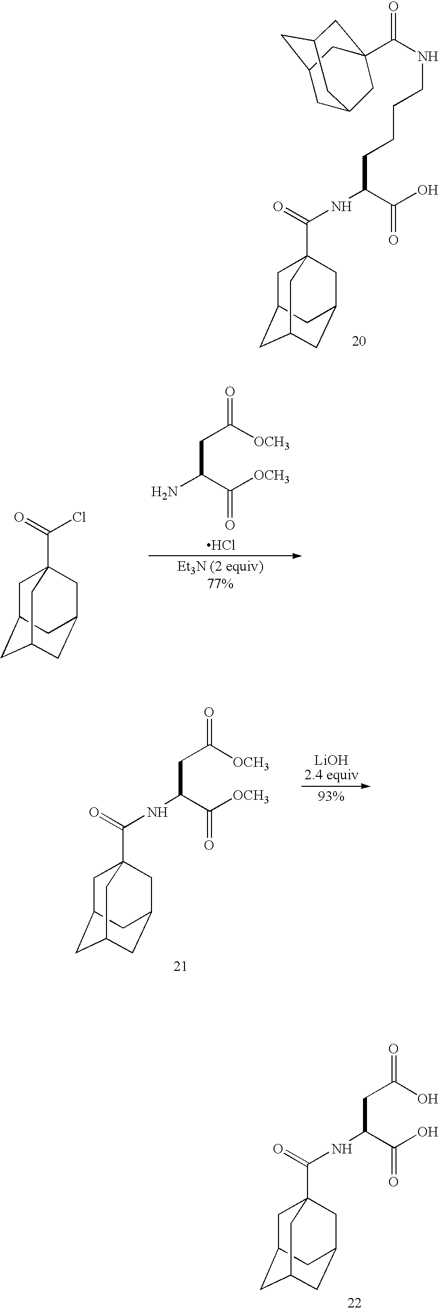

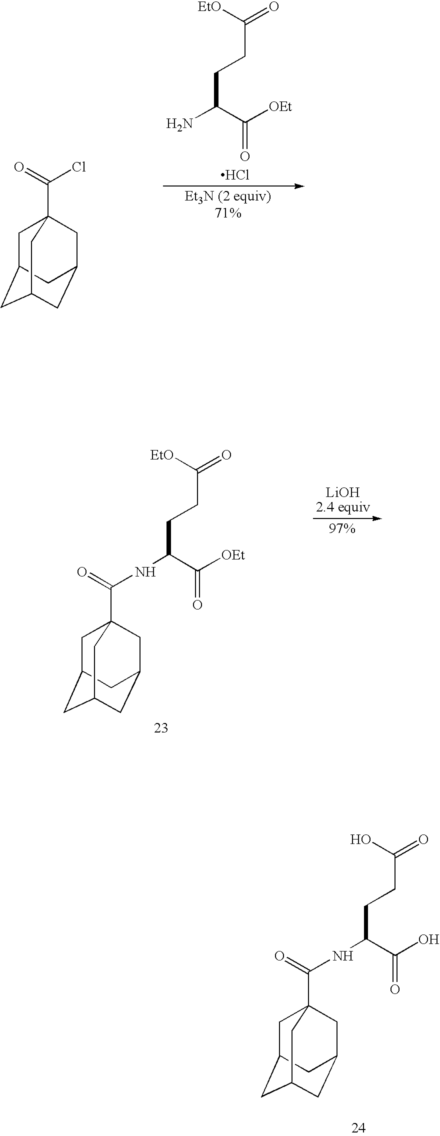

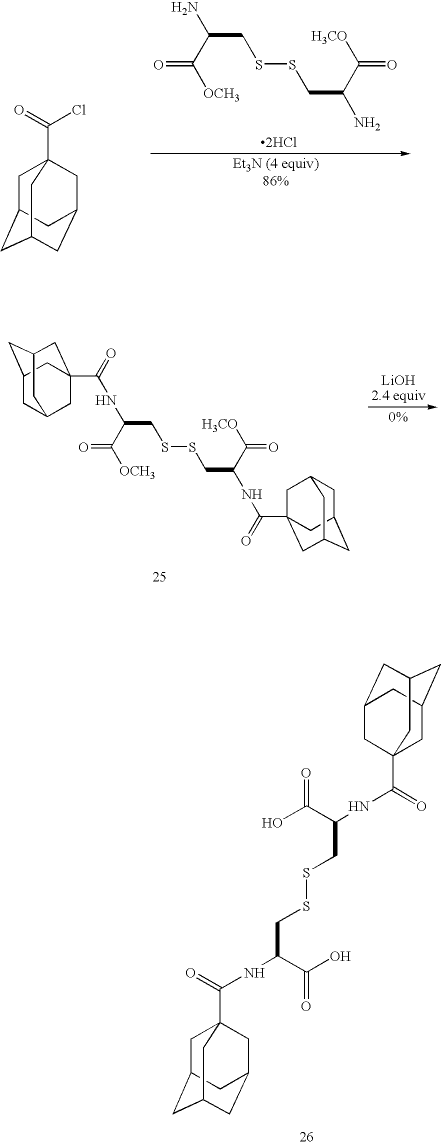

In the instance of the attempted conversion of the N,N-diadamantylcarbonyl-L-cystine methyl ester (25) to the di-acid (26), saponification of 25 with lithium hydroxide resulted in the decomposition of 25. Both 25 and 26 have been reported to be biologically active antagonists for the α4 β1/VCAM cell line with IC₅₀'s of >800 μM and 7.3 μM, respectively. The α4 β1 (VLA-4, very late antigen-4, CD49d/CD29) comprises a group of integrin proteins which are cell surface receptors implicated in mediating cell-cell and cell-matrix interactions as well as processes of inflamation. Antagonists for this family of receptors are being explored as possible treatments for inflammatory and chronic diseases of auto-immune origin. The diacid 26 was successfully synthesized by reacting 1-adamantylcarbonyl chloride with L-cystine under bi-phasic conditions (Scheme 31).

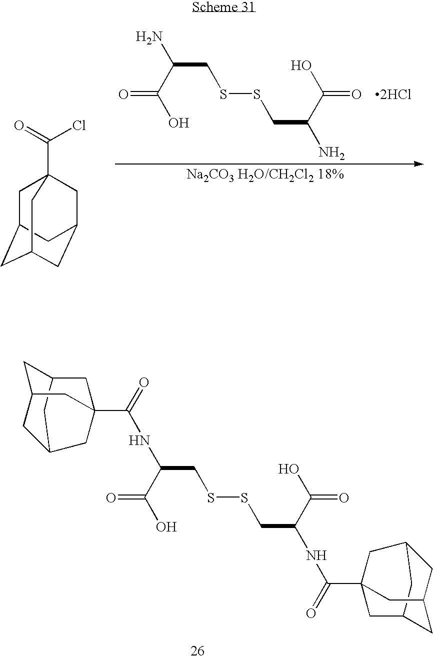

While 25 and 26 represent very simple small molecules with potent antagonist activity for the α4 β1 receptor, the disulphide functionality may not be metabolically robust enough to serve as a small molecule drug. However, the series of additional adamantyl amides 17-26 merit further examination for pharmacaphoric activity, and arrangements for testing 17-26 are in progress.

A variety of 1-boraadamatane amine complexes were synthesized by stoichiometric addition of a primary amine to 1-boraadamantane·THF. These complexes were subsequently evaluated for an antiproliferative effect on CD81-enriched cell lines to provide evidence for binding and activation of the CD81 receptor. Additional amantidine analogues structurally similar to the 1-boraadamantane amine complexes were also synthesized. Several compounds displayed an antiproliferative effect on the CD81 -enriched astrocytes relative to the CD81 -deficient cell lines suggesting that all compounds bind and activate the CD81 receptor over a 0.1 to 10 μM range of concentration. However, analogue 15 displayed the greatest antiproliferative character in this range of concentrations

The analogues 5, 7, 8, 13, 15 and 16 exhibited astrocyte-selective antiproliferation in a concentration range of 0.1 to 10 μM which is indicative of CD81 dependent antiproliferation and establishes the utility of the 1-boraadamantane amine derivatives of the present invention as anti-HCV agents. If this finding proves to be true, such 1-boraadamantane amine derivatives and structurally similar analogues will have the potential to prevent or treat HCV infections by blocking viral attachment, virus-cell fusion or virus entry into host cells, or by countering potential mechanisms of HCV immune evasion. While much remains to be learned about HCV viral attachment and entry, this step in the viral lifecycle has proven to be an effective point of attack for combating other viruses including HIV and rhinoviruses. Molecular modeling also provides a sound theoretical basis and illustration for how the adamantyl framework of 1-boraadamantane amine compounds might bind to CD81. Indeed, the ease with which amines can be appended to the 1-boraadamantane framework makes it an ideal substrate for the quick and convenient synthesis of 1-aminoadamantane analogues for evaluation of potential binding to CD81.

What is claimed is:

1. A method for inducing CD81 dependent antiproliferation in a human or veterinary patient, said method comprising the step of:
(A) administering to the patient a therapeutically effective amount of an amantadine analogue having the formula: 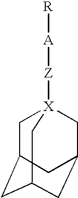
wherein,
X is Boron or Carbon;
A is NH and NHR1, where R1 is H, alkyl or imino-alkyl amino;
Z is a acyclic or cyclic, saturated or unsaturated, chiral or achiral, straight or branched hydrocarbyl group with from 1 to 10 carbon atoms, C═O, SO2, or absent; and
R is an acyclic or cyclic, saturated or unsaturated, chiral or achiral, straight or branched hydrocarbyl group with from 1 to 20 carbon atoms and —CH—R2R3 —CHR2R3, wherein R2 is selected from aryl, substituted aryl, heteroaryl, substituted heteroaryl, (CH2)n-Q, where n is 1-4 and Q is BSH, —OH, —NH2, —NH—CO—NH2, —NH—C═(NR4) NHR5, —COOH and its alkyl esters, and —CONH2 and R4 and R5 are H, C1-4 alkyl or R4 and R5 may combine to form a cyclic ring, R2 and A may combine to form a cyclic ring; R3 is carboxyl, its alkyl esters, carboxamide or substituted carboxamide, sulfonic acid, sulfonate esters, sulfonamide, substituted sulfonamide, phosphonic and phosphoric acids and their alkyl esters.

2. A method according to No. 1 wherein the method is carried out to prevent or treat Hepatitis C.

3. A composition of matter having the formula:
A method for inducing CD81 dependent antiproliferation in a human or veterinary patient, said method comprising the step of:
(A) administering to the patient a therapeutically effective amount of an amantadine analogue having the formula: 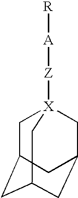
wherein,
X is Boron or Carbon;
A is NH and NHR1, where R1 is H, alkyl or imino-alkyl amino;
Z is a acyclic or cyclic, saturated or unsaturated, chiral or achiral, straight or branched hydrocarbyl group with from 1 to 10 carbon atoms, C═O, SO2, or absent; and R is an acyclic or cyclic, saturated or unsaturated, chiral or achiral, straight or branched hydrocarbyl group with from 1 to 20 carbon atoms and _{—CH—R}2R3, wherein R2 is selected from aryl, substituted aryl, heteroaryl, substituted heteroaryl, (CH2)n—Q, where n is 1-4 and Q is BSH, —OH, —NH2, —NH—CO—NH2, —NH—C═(NR4) NHR5, —COOH and its alkyl esters, and —CONH2 and R4 and R5 are H, C1 -4 alkyl or R4 and R5 may combine to form a cyclic ring, R2 and A may combine to form a cyclic ring; R3 is carboxyl, its alkyl esters, carboxamide or substituted carboxamide, sulfonic acid, sulfonate esters, sulfonamide, substituted sulfonamide, phosphonic and phosphoric acids and their alkyl esters.
